(19) United States
(12) Patent Application Publication
(10) Pub. No.: US 2006/0212129 A1
(43) Pub. Date: Sep. 21, 2006

(54) Partial hand prosthesis

(76) Inventors: Joseph Christopher Lake, Colleyville, TX (US); Michael Alan Ergo, Frisco, TX (US)

Correspondence Address: NAVARRO LAW OFFICE, P.C., P.O. Box 166851, Irving, TX, 75016, US

(21) Appl. No.: 11/081,402

(22) Filed: Mar. 16, 2005

(51) Int. Cl. A61F 2/54; A61F 2/70

(52) U.S. Cl. 623/24; 623/64

(57) ABSTRACT

A prosthetic device (12) and liner (14) form a prosthetic system (10) that can substantially mimic the cosmetic and functional qualities of a human hand. A harness (22) is adapted to fit about a remnant portion (16) of a hand (18). The harness has an opening (26) for accommodating the liner (14). A moveably mounted opposition member (24) is supported by the harness (22) and sensors (60, 62) positioned about the harness (22) detect a force exerted by an area (40) in proximity to the hypothenar eminence of remnant portion. Once a force is detected by either sensor (60, 62) a signal is communicated to a controller (70) which activates a motor (100) causing motion of the opposition member (24).

### TECHNICAL FIELD

The present invention relates generally to a prosthetic device for a hand. More specifically, the invention relates to a partial hand prosthesis that substantially replicates the functionality and look of fingers of a human hand. Still more particularly, the invention provides an electro-mechanical fitted and partial hand prosthetic device that senses motion on an area substantially proximate the hypothenar eminence of the remnant portion of a hand in order to activate the motion of a prosthetic finger member moveably attached to a prosthetic harness surrounding the hand.

### BACKGROUND OF THE INVENTION

Prior attempts have been made to provide a prosthetic device that simulates the look and functionality of the human hand. Most such attempts fall into two camps: cosmetic type prosthesis and active type prosthesis. Cosmetic type prosthetic devices are generally passive and provide no active functionality. Thus, with a cosmetic limb the patient often resorts to using a working limb to accomplish routine tasks. This is not generally desired as it unnatural and can lead to complications associated with overuse syndrome of the working limb.

An active type prosthesis typically comprises a socket mounted to a patient's arm with a coupling attached to the socket. The coupling provides an attachment point for a terminal device or task specific tools and different tools can be used depending on the particular task to be accomplished. Prosthetic devices of this type are functional but not cosmetically pleasing as they generally don't attempt to mimic the look and feel of a real human hand.

A subclass of the active type class of prosthesis is the electro-mechanical type which tries to replicate both the look and function of the human hand. Such devices typically rely on muscle and tendon forces in other parts of the arm in order to activate the grasping function of the fingers. For example, the upper arm muscle may be flexed in order to cause a set of artificial finger member to come together or grasp an item. However, with such prior art electro-mechanical prosthetic devices, the normal physiologically unconscious use of the device is difficult as the patient may need to concentrate the action of the muscles or tendons providing the operating force.

Partial hand amputations where the thumb remains intact present a common type of limb deficiency. Prior art prosthetic devices adapted to such cases pose significant disadvantages due to overall lack of adjustability during use. In most cases, patients pre-position the device with their working hand to place fingers extending from the device in a position accommodating a proposed task. Even when the prosthetic device is designed with an opposition post, i.e. a member that resists the motion of one or more working digits of the hand, the lack of feedback to the patient requires the patient to reposition the device for each new task.

The cases involving partial hand amputations with thumb intact have not, until the present invention, been treated as a special class of limb loss for purposes of prosthetic treatment. No prior art prosthetic is known to effectively utilize the residual thumb and neighboring structures of the remnant portion of the hand in order to help the patient adjust the grip of the device without use of a functional limb.

### BRIEF DESCRIPTIONS OF THE DRAWINGS

The present invention is illustrated by way of example and not limitation in the figures of the accompanying drawings in which like references indicate similar elements, and in which:

FIG. 1 shows a partial hand prosthetic system according to the invention;

FIG. 2 shows is a detailed illustration of a liner which can be used as part of the partial hand prosthetic system of FIG. 1;

FIG. 3 shows the detailed illustration of a prosthetic device which can be used as part of the partial hand prosthetic system of FIG. 1;

FIGS. 4a-4c illustrate the operation of an opposition member with respect to a remnant thumb;

FIG. 5 illustrates the prosthetic system of the present invention on a remnant portion of a hand;

FIG. 6 is a block diagram illustrating the various electro-mechanical components within a prosthetic device according to the invention;

FIG. 7 shows the details of on exemplary embodiment of a drive subsystem used to move one or more prosthetic fingers of a prosthetic device according to the invention; and

FIG. 8 is a detailed mechanical of one exemplary embodiment of a motor, gear head and drive gear arrangement of a prosthetic device according to the invention.

### DETAILED DESCRIPTION

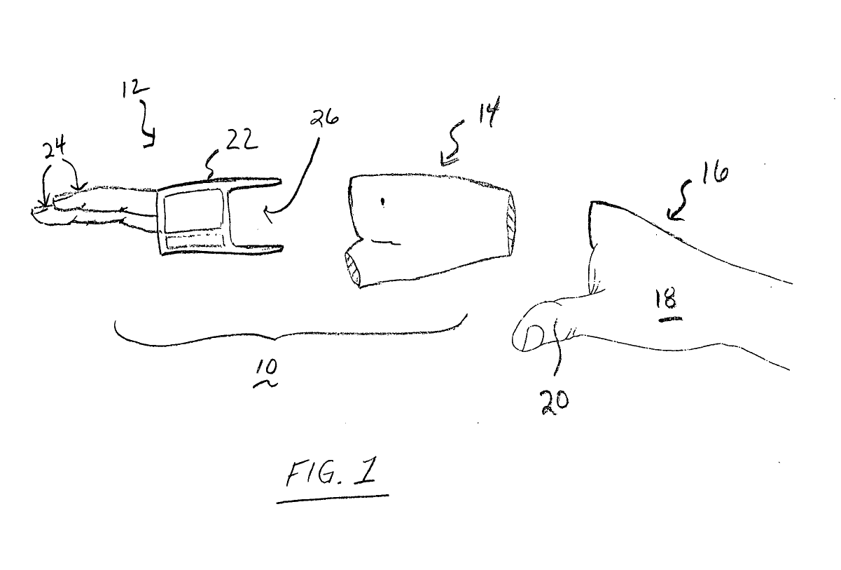

Referring now to the figures, FIG. 1 shows a prosthetic system, denoted generally as 10, consisting of prosthetic device 12 and liner 14 which are designed to fit over a remnant portion 16 of a hand 18. As shown, hand 18 maintains a working thumb thus remnant portion 16 forms a partial hand and prosthetic system 10 is intended to provide a partial hand prosthesis as described herein. In this regard, prosthetic device 12 includes a harness 22 to which one or more prosthetic fingers 24 are movably attached. In a more general sense, prosthetic fingers 24 are designed to move in a direction towards thumb 20 when prosthetic system 10 is worn by a user to a point where it opposes the movement of thumb 20. As such, the terms “prosthetic fingers”, “finger” or “opposition members” can and will be used interchangeably throughout.

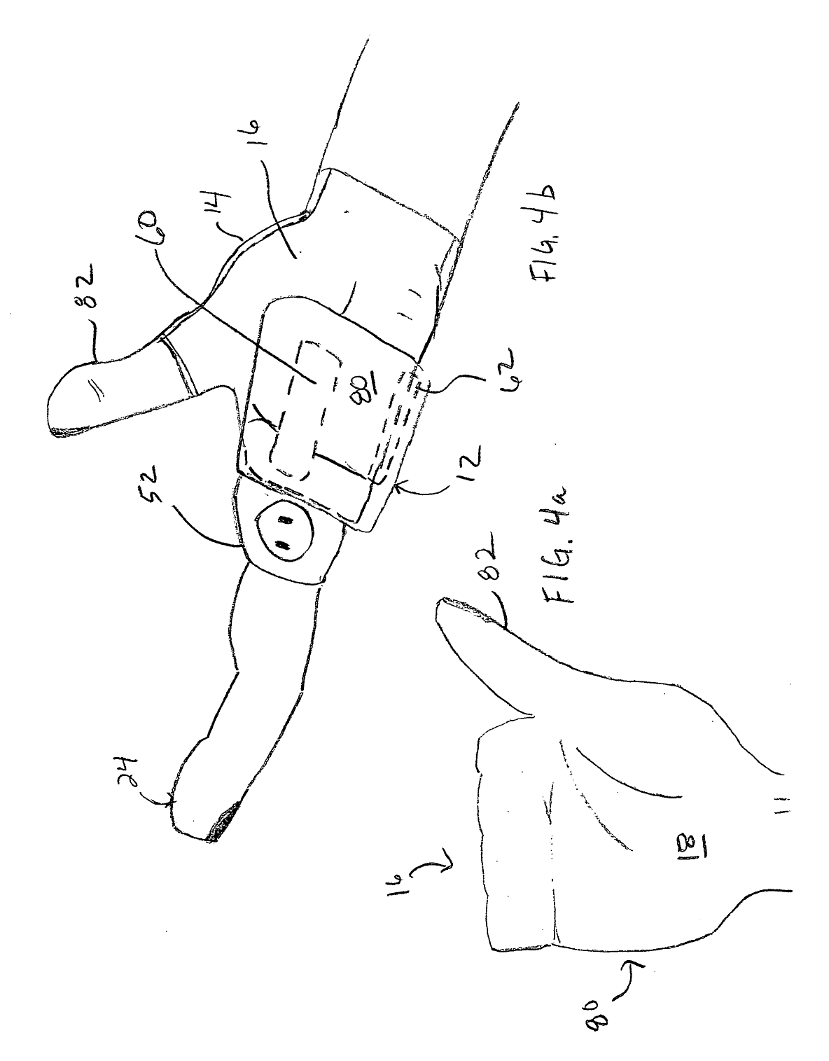

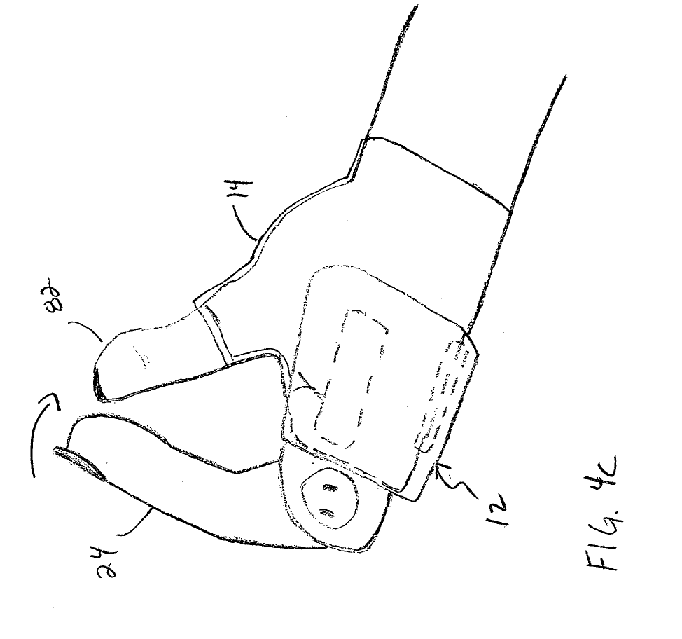

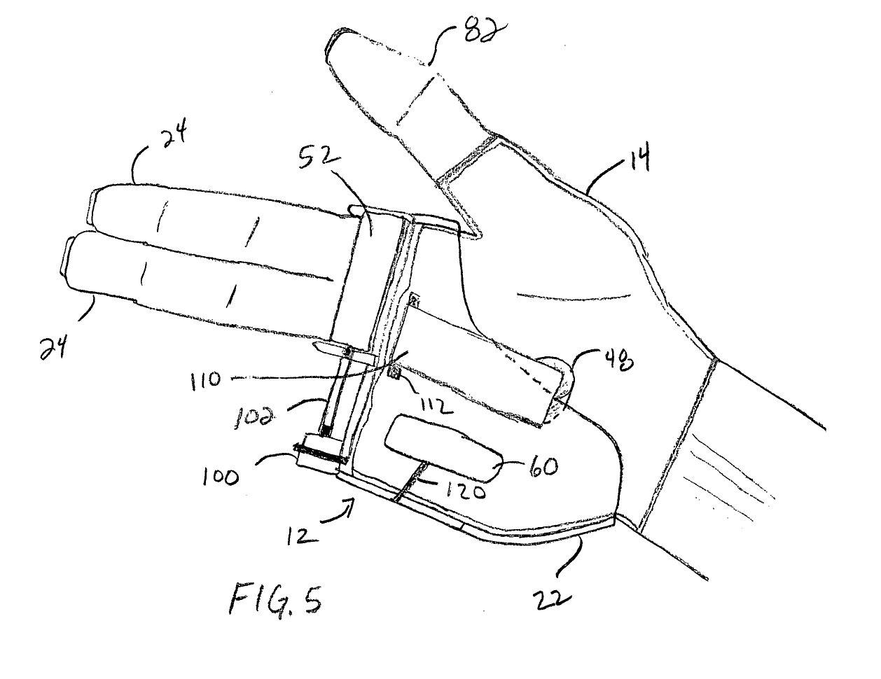

Liner 14 provides a means for attaching prosthetic device 12 to remnant portion 16 of hand 18. Preferably, liner 14 provides a snug slip-free fit over remnant portion 16 to securely hold prosthetic device 12 in place (as shown in FIGS. 4a, 4b and 5). In this regard, it has been found that a silicone liner works well although it is contemplated that a suitable liner can be made from other materials all within the scope of the invention. In any event, as shown, liner 14 is shaped to accommodate prosthetic device 12 about opening 26 which is shaped to accept liner 14 as well as remnant portion 16. FIG. 5 shows the entire prosthetic system 10 worn by a user with remnant portion 16 wearing liner 14 and within opening 26 of the prosthetic device 12.

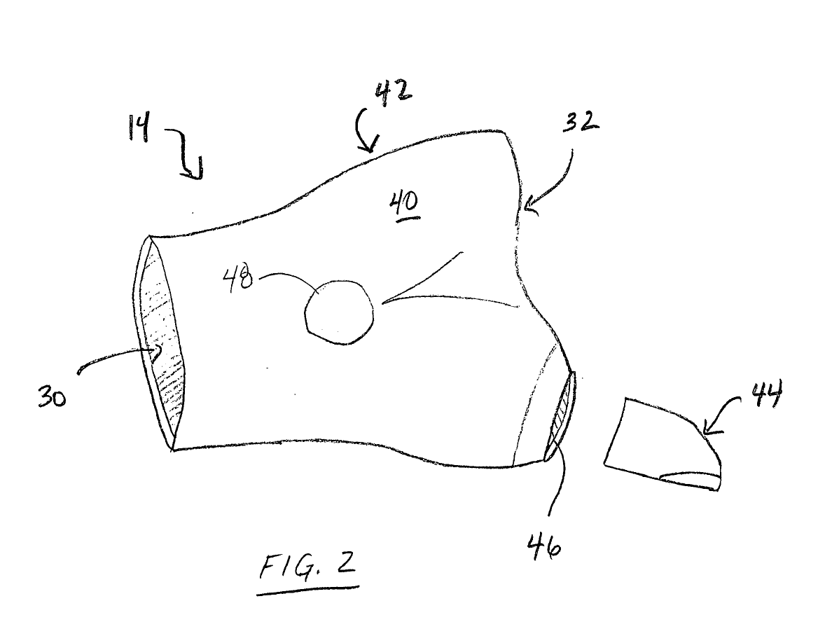

FIG. 2 illustrates further details of liner 14 suitable for use in a prosthetic system according to the invention. Liner 14 provides an opening 30 for inserting the remnant portion 16 of a hand 18 during fitting. Of course, it is contemplated that liner 14 will form a relatively snug fit about a user's hand and that liner 14 including opening 30 and other features thereof may be sized accordingly to accommodate various hand sizes and remnant forms. End periphery 32 of liner 14 is closed forming a glove-like structure for surrounding a remnant portion. In the particular configuration shown, liner 14 includes a second opening 46 for accommodating a thumb. Alternatively, and for cases were a portion of a patient's thumb has been lost, an artificial thumb extender 44 can be attached to the liner 14 to extend opening 46 into a structure substantially approximating a human thumb. Liner also includes areas 40 and 42 which are near an area known as the hypothenar eminence near the lateral portion of a human hand. Finally, liner 14 provides an attachment point 48 to which the prosthetic device 12 can be secured. The invention is not limited to any specific means of attaching the prosthetic device 12 to the liner 14 although it is preferred that prosthetic device 12 be securely attached to the liner 14.

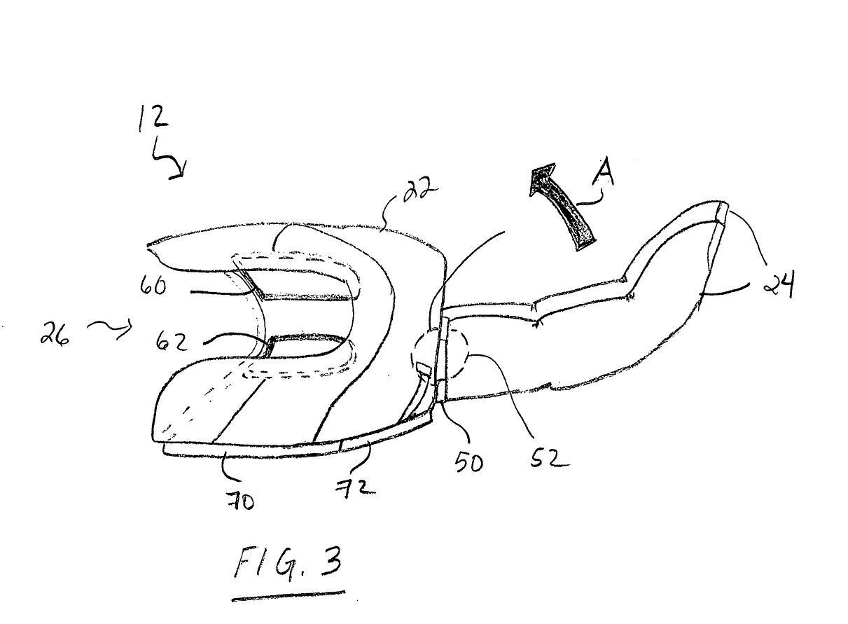

FIG. 3 shows the prosthetic device 12 in more detail. Harness 22 provides a structure to which the various electro-mechanical components for a prosthesis according to the invention can be attached. Opposition members 24 are moveably attached to frame 50 which can rotate about shaft member 52 in the direction of arrow A. Shaft member 52 is provided to allow the rotational motion of the opposition members 24 about harness 22. As shown, attached to harness 22 are sensors 60 and 62 which are positioned about an area where the hypothenar eminence of a remnant portion of a hand inserted into opening 26 of harness 22 would be. Each sensor is communicably attached to a controller, represented by 70, which receives signals from the sensors 60, 62 corresponding to motion of the remnant portion within harness 22. The controller 70 is shown attached to the outside of harness 22 although the controller 70 can likewise be placed in other areas about the harness 22 consistent with the invention. Another block 72 is shown attached to harness 22 and is representative of a power source, such as rechargeable batteries, used to provide energy to the various electro-mechanical components of the prosthetic device 12. This would include controller 70 and motor (not shown in FIG. 3) to drive the shaft member 52 and cause opposition members 24 to pivot about harness in the direction of arrow A.

FIGS. 4a, 4b and 4c illustrate the operation of a prosthetic system, such as prosthetic system 10, according to the invention. In particular, the lateral edge 80 of a remnant portion 16 of a hand 18 is shown. As shown, remnant portion 16 includes a remnant thumb 82 which is functioning normally. In FIG. 4b, the prosthetic device has been fitted over liner 14 which, in turn, has been fit over remnant portion 16. A shaft member 52 is mechanically coupled to the opposition member 24 and operated by a motor (not shown in FIG. 4b). As shown, sensors 60 and 62 are arranged proximate to area 80 which is in the vicinity of the hypothenar eminence of the remnant portion 16. In this position, the sensor 60, 62 are situated to detect the motion of the hypothenar eminence which causes the movement of opposition member 24. The fact that motion of the hypothenar eminence causes motion of opposition members 24 provides a prosthetic device that is physiologically natural for a patient using a prosthetic system according to the invention.

FIG. 4C shows the opposition member 24 moving in the direction of remnant thumb 82 after motion has been activated. This motion more naturally mimics the function of a human finger opposing the thumb during normal use. Thus, the invention provides a prosthetic system that is both functional and cosmetically similar to a human hand.

FIG. 5 shows an alternate view of a prosthetic system according to the invention. Harness 22 of prosthetic device 12 is slipped over liner 14 and held securely in place by strap 110 looping through opening 112 and holding with attachment point 48. Of course, other ways of holding prosthetic device 12 in place may be utilized. Sensor 60 is communicably coupled via signal line 120 to a controller (not shown in FIG. 5) that actuates motor 100 to drive shaft 102 and thereby operate shaft member 52 coupled to opposition members 24. In this way, motion detected by sensor 60 causes the movement of opposition members 24.

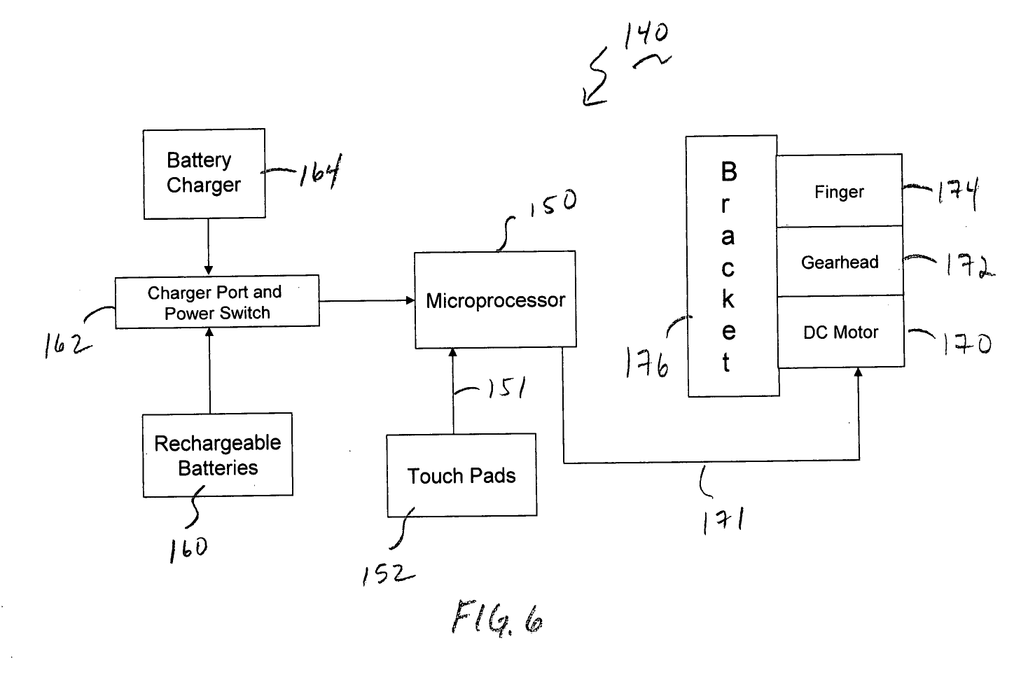

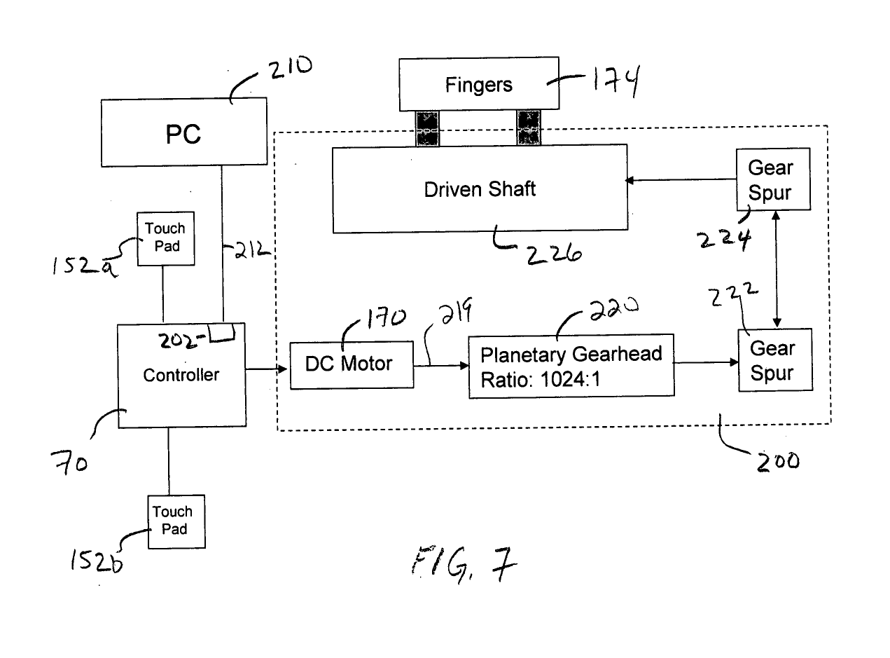

It is contemplated that a variety of electro-mechanical components and device designs may be employed in order to achieve a prosthesis embodying a prosthetic device and system according to the present invention. Therefore, presented herein is but a single embodiment of various electro-mechanical devices and components which have been found to provide the objects and advantages of the invention. As such, FIG. 6 is a block diagram for a prosthetic device, denoted generally as 140, including the various electro-mechanical devices and components according to a general embodiment of the invention. A microprocessor 150 acts as the central processing unit of the prosthetic device 140 and receives an input from touch pads 152 via signal path 151. Touch pads 152 are positioned within a prosthesis structure, such as harness 22, where movements of the hypothenar eminence can be detected and communicated to microprocessor 150. As shown, power is derived from a set of rechargeable batteries 160 which, in combination with charger port and power switch 162 as well as battery charger 164, are capable of providing energy to various electro-mechanical components of the prosthetic device 140.

Prosthetic device 140 also includes a DC motor 170 operably coupled to microprocessor 150 via path 171. Thus, microprocessor 150 may actuate DC motor 170 following the receipt of a detect signal from touch pads 152. DC motor 170 may, in turn, operate gear head 172 to cause the motion of finger member 174. As shown, DC motor 170, gear head 172 and finger 174 are supported by bracket 176.

Referring to FIG. 7, a block diagram for a prosthetic device according to the invention with the details of a drive system, denoted generally as 200, is shown. Specifically, controller 70 is communicably coupled to touch pads 152a and 152b. In addition, controller 70 is provided with port 202 to receive programming instructions from computer 210 via signal path 212. In this way, the controller 70 can be programmed to configure various features of the prosthetic device. For example, computer 210 can be used to set the sensitivity of the touch pads 152a, 152b. In addition, computer 210 can be used to set the direction and speed of the DC motor 170 in order to effect the action of fingers 174. Also, the limit for the stall current of the DC motor 170 can be set. Of course, other variable and features of the prosthetic device can be changed, modified or set using computer 210 though port 202.

As shown, DC motor 170 is in operative communication with planetary gear head 220 which receives a first torque force from the output 219 of DC motor 170 and converts it to a second torque force suitable for driving driven shaft 226 and providing sufficient torque to move fingers 174. This may be accomplished by setting the gear ratio between the input and output of the planetary gear 220 in order to provide a sufficient torque of the driven shaft 226. While various gear ratios can be used, it has been found that a high ratio with respect to the input and out is satisfactory. In particular, according to one specific embodiment, a gear ratio of 1024:1 has been found to provide a sufficient torque force at the driven shaft 226 while preventing back drive of the motor 170. Of course, other ratios may be employed within the scope of the present invention.

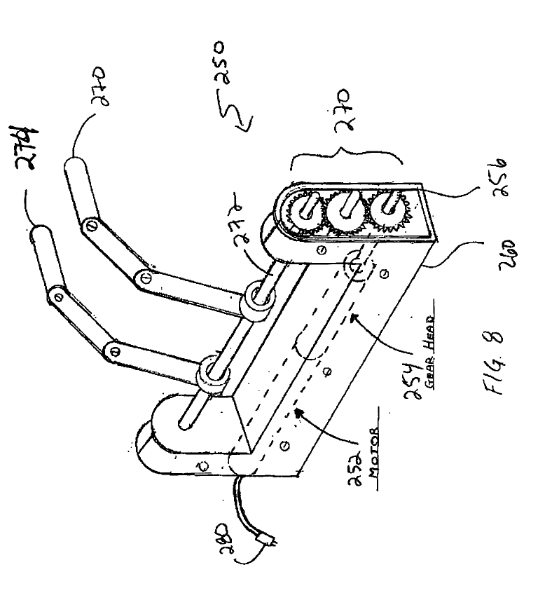

The planetary gear head 220 is operably coupled to gear spurs 222 and 224 which transfer the torque generated by DC motor 170 and gear head 220 to the driven shaft 226 coupled to fingers 174. Other transfer methods may also be employed including, for example, a three gear spur arrangement as shown in FIG. 8. In this way, power provided by the DC motor 170 can be used to move fingers 174. Of course, it is contemplated that variations of the drive subsystem 200 will be apparent to those of ordinary skill in the art and, therefore, such variations should be considered within the scope of the invention.

A mechanical layout of a drive system suitable for use in a prosthetic device according to the invention is shown in more detail in FIG. 8. The drive system, denoted generally as 250, uses a motor 252 to operate gear head 254 along shaft 256. Frame 260 supports these various components as gears 270 transfer mechanical power from the motor 252 to finger members 274 attached to rod 272. A power connector 280 supplies energy from a power source (not shown in FIG. 8) to the motor 252.

As indicated above, various devices and components can be used in order to achieve a prosthetic device having the various features and advantages of the present invention. Table 1 below provides some a material list for some of the electrical and mechanical devices which have been found satisfactory in reducing to practice a suitable prosthesis according to the invention:

TABLE 1
List of parts and part numbers for devices and
components used in an exemplary prosthetic
device according to the invention.

| Part Num | Part Name | Quantity | Vendor |
|---|---|---|---|
| 118396 | Motor, 6 V DC | 1.0 | Maxon |
| 218418 | Gearhead, Planetary Type, | 1.0 | Maxon |
| | 1024:1 | | |
| UPB563450/PCM | Battery, 3.7 Volt, Lithium | 2.0 | Ultralife |
| | Ion | | |
| B84-0500/0550 | Battery Charger, Lithium | 1.0 | Energy |
| | Ion, 1000 mA | | Access |
| 910-2050-05 | Index Finger | 2.0 | MIL-E |
| 970-2060-01 REV C | Hand Mounting Assembly | 1.0 | MIL-E |
| 970-2060-10 REV B | Gear Cover | 1.0 | MIL-E |
| 970-2060-20 | Motor Shaft | 1.0 | MIL-E |
| 970-2060-30 | Bearing | 2.0 | MIL-E |
| 970-2060-05 REV C | Axle | 1.0 | MIL-E |
| KJ08412K | Tie Wrap | 1.0 | Dennison |
| FIT-221B, 3/32 | Heat Shrink, Black | 1.0 | Alpha |
| 16P-3100 | Charging Jack | 1.0 | Mouser |
| 910-2020-01 | Power Shutoff Plug, | 1.0 | MIL-E |
| | Modified | | |
| N/A | Mounting Screws, | 4.0 | Global |
| | #4-40 × .125, PFH, SS | | |
| N/A | PlateScrews, | 4.0 | Global |
| | #6-32- × .125, PPH, SS | | |

It should be understood that modifications can be made to the invention in light of the above detailed description. The terms used in the following claims should not be construed to limit the invention to the specific embodiments disclosed in the specification and the claims. Rather, the scope of the invention is to be determined entirely by the following claims, which are to be construed in accordance with established doctrines of claim interpretation.

What is claimed is:

1. A partial hand prosthetic system compromising:
a liner for surrounding the remnant portion of a hand;
a harness adapted to fit about said remnant portion of said amputated hand and having an opening for accommodating said liner;
at least one moveably mounted opposition member supported by said harness;
at least one sensor positioned about said harness and arranged to detect a force exerted by an area of said remnant portion;
wherein a force exerted by said remnant portion of said amputated hand is detected by said sensor and causes said opposition member to move.

2. The partial hand prosthetic system of claim 1 further comprising a power source for providing electro-mechanical power to said opposition member.

3. The partial hand prosthetic system of claim 2 further comprising an electric motor supported by said harness for causing said opposition member to move in combination with said power source.

4. The partial hand prosthetic system of claim 3 wherein further comprising a controller communicably coupled to said sensor and having an output connected to said electric motor which drives said electric motor to cause said opposition member to be actuated.

5. partial hand prosthetic system of claim 1 wherein said sensor comprises a first touch pad in communication with said controller, said touch pad in contact through said liner with some portion of the hypothenar eminence of the remnant portion of said amputated hand.

6. The partial hand prosthetic system of claim 1 wherein said sensor comprises first and second touch pads in communication with said controller, said touch pad in contact through said liner with some portion of the hypothenar eminence of the remnant portion of said amputated hand.

7. The partial hand prosthetic system of claim 1 wherein said liner comprises a silicone liner that fits over said portion of said amputated hand and is held in place with a suction force.

8. The partial hand prosthetic system of claim 1 further comprising a thumb extension coupled to said liner.

9. The partial hand prosthetic system of claim 1 further comprising a second opposition member.

10. A partial hand prosthesis comprising:
a harness having an opening with a shape accommodating the remnant portion of a hand;
at least one moveably mounted opposition member supported by said harness;
an electro-mechanical subsystem configured to cause said opposition member to move in response to a movement of an area substantially proximate the hypothenar eminence of said remnant portion of said amputated hand.

11. The partial hand prosthesis of claim 10 wherein said electro-mechanical subsystem comprises:
a first touch pad positioned about said harness to make contact with said area substantially proximate the hypothenar eminence;
a controller in communication with said first touch pad; and
a motor having an input operably coupled to said controller, said motor in coupling communication with said opposition member for controlling its motion;
wherein said area substantially proximate the hypothenar eminence makes contact with said first touch pad which signals said controller and wherein said controller causes said motor to move said opposition member.

12. The partial hand prosthesis of claim 11 further comprising a second touch pad about said harness opposite said first touch pad.

13. The partial hand prosthesis of claim 12 wherein said first touch pad detects movements from said area in a first direction and wherein said second touch pad detects movements from said area is a second direction.

14. The partial hand prosthesis of claim 13 wherein said first and second directions correspond to closing and opening movements of said opposition member with respect to a thumb on a remnant portion of a hand.

15. The partial hand prosthesis of claim 10 wherein said opening of said harness accommodates a liner between said harness and said remnant portion.

16. The partial hand prosthesis of claim 10 further comprising a second moveably mounted opposition member supported by said harness.

17. The partial hand prosthesis of claim 11 wherein said electro-mechanical subsystem further comprises a power source providing energy to said motor for moving said opposition member.

18. The partial hand prosthesis of claim 17 wherein said electro-mechanical subsystem further comprises a planetary gear head for converting torque generated by said motor into a sufficient amount of torque for moving said opposition member.

19. The partial hand prosthesis of claim 18 further comprising gears coupling the output of said planetary gear head to a shaft coupling said opposition member to said harness.

20. In connection with a partial hand prosthetic device having at least one finger member with one end rotatably mounted to a shaft member, a prosthetic drive control system comprising:
a motor having an input for initiating a torque drive and an output;
a drive subsystem coupling said motor to said shaft member, said drive subsystem operably connected to said output of said motor for coupling a force to said shaft member that controls the motion of said prosthetic finger member; and
sensor for causing the actuation of said motor, said sensor adapted to detect the movement of an area substantially proximate the hypothenar eminence of a remnant portion of a hand.

21. The prosthetic drive control system of claim 20 further comprising a controller communicably coupled to said sensor and said motor for causing said drive subsystem to control the motion of said prosthetic finger member.

22. The prosthetic drive control system of claim 20 wherein said sensor comprises first and second touch pads sensitive which can be made to be sensitive to the hypothenar eminence of a hand.

23. The prosthetic drive control system of claim 20 wherein said drive subsystem comprises a planetary gear head for converting a first torque force received from said motor over said output to a second torque force.

24. The prosthetic drive control system of claim 23 wherein said drive subsystem further comprises first and second drive gears coupling said planetary gear head to said shaft member.

25. The prosthetic drive control system of claim 23 wherein said drive subsystem further comprises first, second and third drive gears coupling said planetary gear head to said shaft member.

26. The prosthetic drive control system of claim 24 wherein said planetary gear head substantially eliminates back drive.

27. The prosthetic drive control system of claim 20 wherein said motor is a DC motor and further comprising a power source for providing energy to said DC motor.

28. The prosthetic drive control system of claim 21 wherein said controller further comprises a computer port for setting a variety of prosthetic function variables.

29. The prosthetic drive control system of claim 28 wherein said prosthetic function variables are selected from the group consisting of: the sensitivity of said sensor, the direction and/or speed of said motor and the limitation of stall current related to said motor.

30. In connection with a partial hand prosthesis having an opposition member moveably attached to prosthetic harness, the prosthetic harness providing a platform for a plurality of electro-mechanical components, a method of causing said opposition member to move comprising the steps of:
a sensor detecting the movement in a first direction of an area substantially proximate the hypothenar eminence of a remnant portion of a hand;
the sensor communicating a signal to a controller indicating movement has been detected;
the controller activating a motor operably coupled to said opposition member through a drive subsystem;
the drive subsystem transferring a force from said motor to said opposition member thereby causing said opposition member to move in a first direction towards a thumb of said amputated hand.

31. The method of claim 30 wherein the step of said drive subsystem transferring a force is performed by the drive subsystem converting a torque force from the motor to a greater torque driving a said of gears coupled to said opposition member.

32. The method of claim 30 further comprising the step of the sensor detecting the movement in a second direction of an area substantially proximate the hypothenar eminence of a remnant portion of a hand.

33. The method of claim 32 further comprising the step of the drive subsystem transferring a force from said motor to said opposition member thereby causing said opposition member to move in a second direction away from a thumb of said amputated hand.
